(19) United States
(12) Patent Application Publication
(10) Pub. No.: US 2026/0318913 A1
(43) Pub. Date: Oct. 15, 2026

(54) PORTABLE DETECTION SYSTEM FOR DETECTING A PATHOGEN OR VIRUS

(71) Applicant: THE UNIVERSITY OF SYDNEY, Sydney (AU)

(72) Inventor: Ken Tye YONG, Sydney (AU)

(21) Appl. No.: 19/481,243

(22) PCT Filed: Apr. 30, 2024
(86) PCT No.: PCT/AU2024/050417

§ 371 Date: Nov. 4, 2025

(30) Foreign Application Priority Data
May 8, 2023 (AU) 2023901382

(51) Int. Cl. G01N 33/543; G01N 21/77; G01N 33/569

(52) U.S. Cl. CPC G01N 33/54393; G01N 21/7703; G01N 33/569; G01N 2021/7709; G01N 2201/0221; G01N 2333/255

(57) ABSTRACT

A portable detection system for detecting a pathogen or virus, the portable detection system comprising: at least one light source for generating an input optical signal; at least one optical sensor comprising at least one optical fibre having at least one tapered sensing region with at least one coating that reacts with a pathogen being detected, wherein the optical sensor is arranged to receive the input optical signal, communicate the input optical signal through the tapered sensing region and communicate an output optical signal from the tapered sensing region; at least one photodetector for detecting power intensity of the output optical signal, wherein the photodetector is arranged to generate an output signal based on a change in power intensity of the output power at a resonant wavelength that corresponds with the pathogen; and at least one microcontroller for controlling the light source, optical sensor and photodetector; wherein the portable detection system is arranged to cause an output to be generated based on the output signal, wherein the output is associated with the pathogen being detected.

### TECHNICAL FIELD

The present invention relates generally to a portable detection system for detecting a pathogen or virus and an associated detachable probe.

### BACKGROUND

Systems and methods for detecting pathogens or viruses, including methods such as PCR techniques and DNA sequencing, and systems such as automated microbial identification systems and immuno-diagnostic assay systems, require large and expensive instrumentation that is generally not suitable for on-site measurements.

### SUMMARY

It is an object of the present invention to substantially overcome, or at least ameliorate, one or more disadvantages of existing arrangements.

Disclosed are arrangements which seek to address the above problems by providing a portable detection system utilising an optical system that is used for determining a change in power intensity of output power at a resonant wavelength that corresponds with the detection of a pathogen or virus.

According to a first aspect of the present disclosure, there is provided a method for a portable detection system for detecting a pathogen or virus, the portable detection system comprising: at least one light source for generating an input optical signal; at least one optical sensor comprising at least one optical fibre having at least one tapered sensing region with at least one coating that reacts with a pathogen being detected, wherein the optical sensor is arranged to receive the input optical signal, communicate the input optical signal through the tapered sensing region and communicate an output optical signal from the tapered sensing region; at least one photodetector for detecting power intensity of the output optical signal, wherein the photodetector is arranged to generate an output signal based on a change in power intensity of the output power at a resonant wavelength that corresponds with the pathogen; and at least one microcontroller for controlling the light source, optical sensor and photodetector; wherein the portable detection system is arranged to cause an output to be generated based on the output signal, wherein the output is associated with the pathogen being detected.

According to another aspect of the present disclosure, there is provided an apparatus for implementing any one of the aforementioned methods.

Other aspects are also disclosed.

### BRIEF DESCRIPTION OF THE DRAWINGS

At least one embodiment of the present invention will now be described with reference to the drawings and appendices, in which:

FIG. 1 shows a tapered fibre sensor according to the present disclosure;

FIG. 2 shows an example of a phage physical adsorption system according to the present disclosure;

FIG. 3 shows a phage-based tapered fibre for detecting at least one pathogen according to the present disclosure;

FIG. 4 shows a schematic diagram of a portable detection system according to the present disclosure;

FIG. 5 shows a schematic diagram of a portable detection system in use according to the present disclosure;

FIG. 6 shows an example of an optical power output signal generated by a portable detection system according to the present disclosure.

FIG. 7 shows an example of the power intensity calibration curve based on the concentration of the pathogen according to the present disclosure.

### DETAILED DESCRIPTION INCLUDING BEST MODE

The example provided herein describes a portable detection system for detecting a pathogen. It will be understood that the same principle can also be applied to detecting a virus.

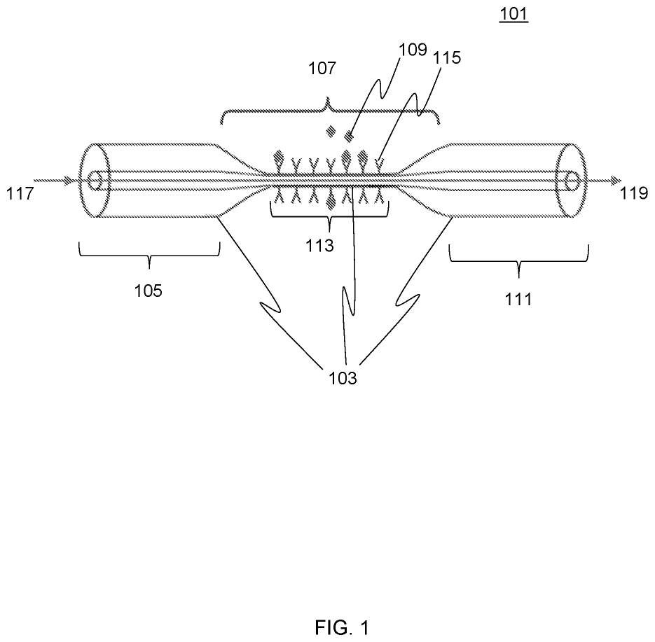

FIG. 1 shows a tapered fibre optical sensor with a sensing region for detection of one or more pathogens, according to one example.

The optical sensor 101 is formed from an optical fibre 103 that has a first non-tapered region 105, a tapered sensing region 107 for sensing a pathogen 109 (shown as diamonds in FIG. 1) and a second non-tapered region 111. The tapered sensing region 107 has at least one coating 113 that is selected from a group of coatings, where the selected coating reacts with the pathogen that is to be sensed by the optical fibre 103 of the optical sensor 101. The coating 113 includes phages 115 for detecting the pathogen.

The optical sensor 101 is arranged to receive an input optical signal 117, communicate the input optical signal 117 through the tapered sensing region 107 and communicate an output optical signal 119 from the tapered sensing region 107.

According to one example, a phage-functionalized tapered fibre may be used as an optical sensor to detect the presence of foodborne pathogenic bacteria. This may be achieved by monitoring the changes in the refractive index of the fibre when a specific bacterium is selectively captured by the corresponding phages functionalized on the fibre sensing surface in the sensing region of the optical sensor. Bacteriophages are small viruses that are harmless to humans and animals, are less expensive and faster to produce with longer shelf life than anti-bodies. Moreover, bacteriophages exhibit thermal stability and can be easily immobilised onto fibre by a simple dip coating technique, for example.

According to one example, a standard SMF-28 fibre was tapered using the glass processing machine, Vytran GPX-3000. The taper profile of the optical microfiber was fixed at 3 mm of down taper length, 2 mm of up taper length, 6 mm taper length, and 7.9 μm of taper waist diameter. The reproducibility of the optical microfiber was verified through the free spectral range (i.e., the spacing between two successive peaks in the output spectrum of the OM) and the extinction ratio (i.e., the 10 log Pₚₑₐₖ/P_{valley}, Where Pₚₑₐₖ and P_{valley} are the optical power at the maxima (peak) and minima (dip) of the output spectrum) and were consistently within 15.5±1.5 nm and 10±0.5 dB respectively.

Other examples of how the taper of the probe may be biochemically treated to create a sensing region are now provided. The immobilization of bacteriophage receptors on the fibre sensing surface in the sensing region of the optical sensor is important to develop phage-based biosensors. The immobilization steps may include, for example, physical adsorption, covalent attachment, and genetic modification of receptors.

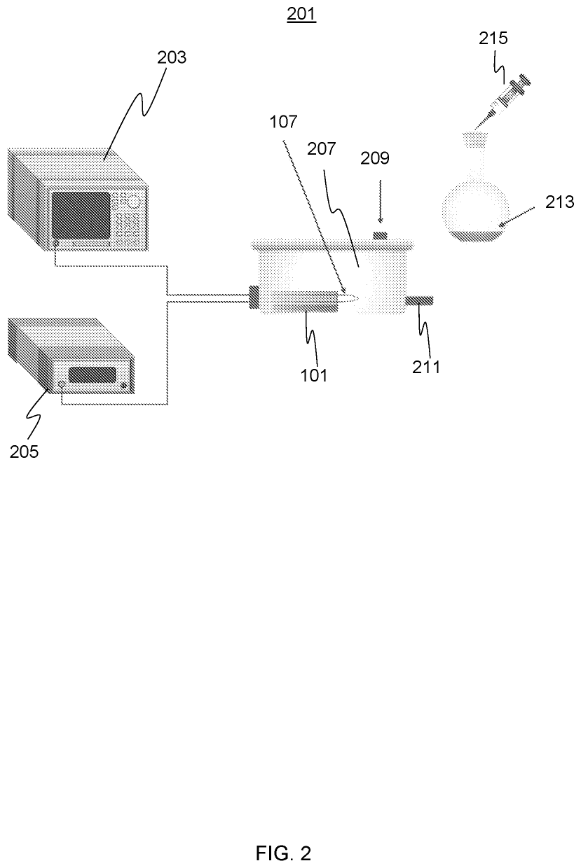

According to one example, the physical adsorption of the phages onto the fibre sensing surface in the sensing region of the optical sensor may be formed by the phage physical adsorption system shown in FIG. 2. The system 201 has an optical spectrum analyser 203 and super luminescent light emitting diode 205 both connected via optical fibre to the sensor 101 having a tapered sensing region 107. The sensor 101 is placed inside a gas sensing chamber 207 that has a gas inlet 209 and a gas outlet 211. Gas is collected from a gas container 213 and then injected into the gas sensing chamber 207, e.g. using a gas syringe 215. This method is described in more detail by Yap et al., Two Dimensional MoS2 Nanosheet-Functionalized Optical Microfiber for Room-Temperature Volatile Organic Compound Detection, ACS Appl. Nano Mater., 2021, 4, 13440-13449, which is hereby incorporated by reference in its entirety. This approach may result in weak binding and an inconsistent number of phages on the surface. Nonetheless, this method has been commonly used for pathogen detection mainly due to the simplicity of the immobilization process. Other methods will also be known to the person skilled in the art.

According to another example, chemically anchoring of the phages to the fibre sensing surface in the sensing region of the optical sensor may be utilised to improve the coverage density significantly on the sensing surface. For example, a cysteine modified gold surface substrate can deposit 7-fold more phages when compared to the physical adsorption on bare gold surfaces and the density of phages improves to 37-fold when the cysteine modified gold surface is activated by a bi-linker such as glutaraldehyde. According to this example, a two-step cysteine-glutaraldehyde-based silica surface chemistry process may be used. The succinimidyl group is then used for chemical linkage to the amine group on the surface of the phages. This method offers straightforward integration into biosensor platforms, and the detection limit of the target bacterium can be achieved to be as low as 102 cfu mL-1.

According to another example, phages may be genetically modified to introduce functional ligands for enhancing the immobilization process onto the fibre sensing surface in the sensing region of the optical sensor. For example, Gervais et al. in Immobilization of biotinylated bacteriophages on biosensor surfaces, Sensors and Actuators B 125 (2007) 615-621 immobilized biotin expressing T4 phages onto gold surfaces by using a monolayer of biotin on the surface and adding streptavidin as the linker. Biotin expressing phages may be immobilized on streptavidin-coated magnetic beads and these beads may be used to capture 72 to 99% of the target bacteria from the suspension for analysis and detection.

The above described examples may be employed to functionalize the tapered fibre for specific sensing of bacteria on food.

Other examples of coatings that may be applied to the fibre sensing surface in the sensing region of the optical sensor for other detection purposes include: (i) nanomaterials (e.g. 2D nanomaterials); (ii) copper complex; (iii) proteins; and (iv) enzymes, which could be used for gas sensing, chemical sensing in liquid, diseased biomarkers detection, harsh environmental monitoring of toxic gas or liquid, saliva screening of diseases, infrastructure security monitoring, etc. It will be understood that the 2-dimensional (2D) nanomaterial may be a single-atom thickness material depending on the reactivity that is required to set up in the optical sensor system. Further, it will be understood that multiple single-atom layers may be arranged to form the material.

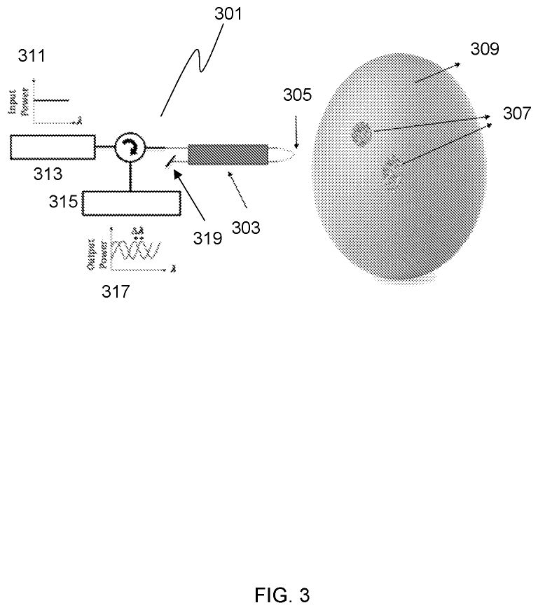

Embodiments of the invention include the fabrication, characterisation and functionalization of a tapered optical sensor with a tapered sensing region, and evaluating the performance of the optical sensor in terms of signal sensitivity and reproducibility, and its ability to perform quantitative analysis of bacteria on food products. A portable detection system incorporating the phage-based tapered fibre sensor can be developed such that rapid on-site testing can be carried out (see FIG. 3 for example).

FIG. 3 shows a phage-based tapered optical fibre sensor for sensing bacteria, such as Salmonella.

An optical probe 301 can be formed using an optical sensor 303 (e.g. the optical sensor 101) that has a biochemically treated tapered sensing region 305, the coating on which has active binding sites to the pathogen 307 that is to be detected. For example, the tapered sensing region 305 can be treated with E2 phage that has active binding sites to detect the pathogen 307 which in this example is Salmonella typhimurium located on an eggshell 309 of an egg. An optical signal (represented by graph 311 showing wavelength vs input power) is generated by an optical light source 313, such as a laser, that is located inside the probe 301, and a photodetector 315 within probe 301 is used to sense a change in the power intensity (represented by the graph 317) of the resonant wavelength. The change in power intensity at the resonant wavelength occurs when the binding of pathogen occurs. According to this example, the optical fibre terminates with a mirror 319 at the end of the optical path to feed the signal back through the optical fibre.

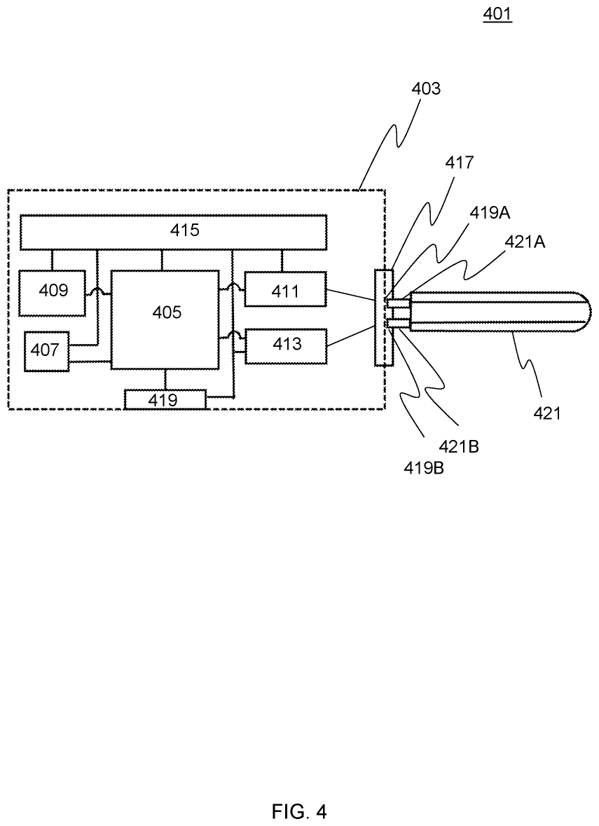

FIG. 4 shows a schematic diagram of an example of a portable detection system 401 as described herein.

The portable detection system 401 has a housing 403 that contains at least one microcontroller 405, at least one memory 407, at least one communications module 409, at least one optical light source 411, at least one photodetector 413, at least one power source 415, at least one sensor interface 417 for connecting with an optical sensor, and an optional output device 419. In this example, the optical sensor is integrated inside a connectable & detachable optical probe 421.

The optical probe 421 could be constructed from an optical sensor, such as the optical sensor 101 described with reference to FIG. 1.

The optical probe 421 may have a sensor interface for interfacing with the portable detection system's sensor interface 417. For example, this may be at least one, two or more optical fibre connections (e.g. (421A, 421B)). +

The optical probe 421 may be a one-time use probe, which may be discarded after one use.

The portable detection system can be arranged to generate the detection results based on the optical output signal that is associated with the pathogen being detected. For example, the detection results may be generated by the portable detection device itself, or by one or more other devices that may be connected to the portable detection device.

In one example, the detachable optical probe may incorporate at least one light source for generating the input optical signal.

In one example, the detachable optical probe may also have at least one photodetector for detecting output power of the output optical signal, wherein the photodetector is arranged to detect a change in power intensity of the output power at a resonant wavelength that corresponds with the pathogen.

The portable detection system 401 may, optionally, have at least one output interface module 419 to enable an output of the pathogen detection process to be provided locally. For example, the output interface module may be arranged to generate one or more of a visual, audible and haptic output based on an output optical signal that relates to whether or not the pathogen has been detected.

The housing 403 can be a unitary housing or maybe formed from multiple sections. The housing may be formed from any suitable material, such as a durable hard plastic.

Any suitable microcontroller or microprocessor can be used, such as an Arduino-type microcontroller that can be used to measure the optical signal from the photodetector and convert analogue signals to digital signals. The same or different microcontroller may be used for Wi-Fi communications, e.g. Arduino Uno or an Arduino IoT module microcontroller.

The memory 407 may be any suitable memory for permanent or semi-permanent storage of program instructions for use by the microcontroller, as well as semi-permanent or non-permanent storage of data.

The communications module 409 can be, for example, a Wi-Fi module for connecting to a local Wi-Fi node to enable the communications module 409 to communicate with a server on the Internet under instructions from the microcontroller 405. The communications module 409 may be, for example, a Bluetooth module for sharing data using Bluetooth communication protocols. Other suitable wireless communication protocols may also be used. The communications module may be used to transmit data to and receive data from one or more other devices such as a mobile telephone, a tablet device, a laptop, a computer, a server, and any suitable smart ware (e.g. a smartwatch, smart glasses etc.) etc.

A software application (“App”) may be used to access the data as well as to retrieve, obtain, transfer, store, display, share or otherwise manipulate data and/or pathogen detection outputs to and from the devices. The App may provide functionalities to display an outcome of the pathogen detection process and to show past, present and/or historical results of the pathogen detection process.

The optical light source 411 may be a laser, photodiode or any other suitable light source. For example, the optical light source may be a fibre coupled super luminescent diode, such as, for example, a diode that provides an output of 15 mW @ 780 nm. One such example is QSDM-780-15D available from QPHOTONICS, LLC.

The photodetector 413 may be any suitable photodetector, such as for example, photodiodes or fibre-based photodetectors produced by Thorlabs, Inc., mini or micro-spectrometers produced by Hamamatsu Photonics K.K., or compact spectrometers produced by Ocean Insight.

As mentioned above, there is a correlation between the power intensity determined by the optical output signal from the optical sensor and the concentration of the pathogen being sensed (e.g.Salmonella). Therefore, a reading of the concentration of the pathogen can be determined from the measured/determined power intensity. The microcontroller of the portable detection system may determine the power intensity at the resonant wavelength based on the signal generated by the photodetector. Alternatively, a separate device in communication with the portable detection system (via communication modules in the portable detection system and separate device) may determine the power intensity at the resonant wavelength based on the signal generated by the photodetector in the portable detection system.

The power source 415 may be battery and rechargeable, for example. The battery is selected to provide sufficient power to all of the components of the portable detection system.

The sensor interface 417 may have a first port 419A and a second port 419B for receiving a first optical fibre connection 421A and a second optical fibre connection 421B, respectively, of a connectable/detachable optical probe 421.

The output device 419 may output, for example, audio, visual, haptic or any other suitable output signal to indicate whether a pathogen has been detected For example, the detection of a pathogen can be indicated by an audio output signal such as a warning beeping sound, or a visual output signal that switches on an LED (e.g. red LED), and a haptic output signal that generates a vibration using a small motor to indicate detection of a pathogen.

The optical signals captured by the optical sensor are processed to determine whether a pathogen is present or not, as described herein. The data processing associated with the captured optical signals may be performed locally if there is sufficient power for the microcontroller to do so. Alternatively, or in addition, the data processing may be performed in any suitable device that can communicate with the communication module (e.g. a smart phone, tablet, laptop, desktop computer, server, smart ware etc.).

Alternatively, or in addition, the data processing may be performed in the App or software program that can analyse the data running in any suitable electronic device, such as a smartphone, tablet, laptop computer, desktop computer or the like.

In one example, the microcontroller in the portable detection system may be arranged to generate the result based on the output signal generated by the photodetector, which detects the output power of the output optical signal. The output interface module may be arranged to generate one or more a visual, audible, and haptic output based on that output.

In another example, the portable detection system (with or without an output interface module) may have a communication module that is arranged to communicate a receiving device signal associated with the output to at least one receiving device. For example, the receiving device may be any suitable device that can communicate with the communication module (e.g. a smart phone, tablet, laptop, desktop computer, server, smart ware etc.

Further, in the portable detection system the receiving device may be arranged to receive the receiving device signal, and based on the receiving device signal, generate one or more of a visual, audible and haptic output based on the output. That is, the receiving device may have a receiving device output interface module for generating one or more of a visual, audible and haptic output based on the output.

In another example, the portable detection system may have an output interface module and a communication module that may be in communication with a connected device (e.g. a further receiving device) to enable the connected device to process the output. For example, the communication module may be arranged to communicate a receiving device signal associated with the output to a further receiving device, and subsequently receive a signal from the further receiving device, wherein that signal is based on the further receiving device determining whether or not the pathogen has been detected based on the receiving device signal. The receiving device and further receiving device may be different devices or the same device. The output interface module may be arranged to generate, based on the received signal, one or more of a visual, audible and haptic output based on the output.

The receiving device signal may include, for example, a set of data based on the output power detected by the photodetector, the power intensity detected by the photodetector, or any other suitable data associated with the photodetector that may be used by the further receiving device to determine whether a pathogen is present or not.

The receiving device signal may include, for example, a binary indication of whether a pathogen is present or not, or any other suitable data associated with the determination of whether a pathogen is present or not to enable the portable detection system to indicate the results of the test.

The output that is generated may be a simple binary output, such as a “Yes” or “No” indicating that a pathogen is present or not respectively. The output may be a colour output, such as a red display or light or a green display or light to indicate that a pathogen is present or not respectively. The output may be an audio output, such as a loud “buzzing” sound or a quiet “ding” sound to indicate that a pathogen is present or not respectively. The output may be a vibration or a non-vibration of a haptic device to indicate that a pathogen is present or not respectively.

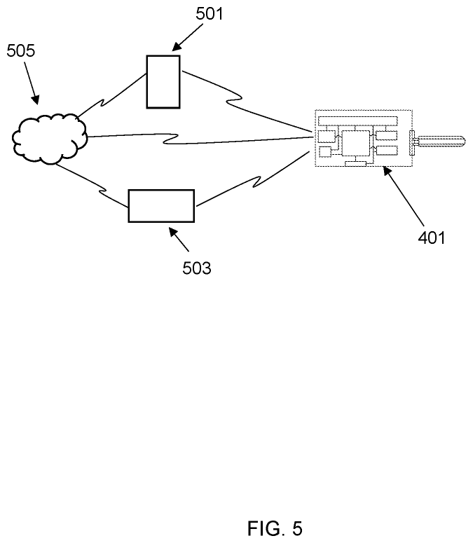

FIG. 5 shows a schematic diagram of a portable detection system in use.

In this example, a portable detection system 401 communicates with a receiving device 501 and/or a further receiving device 503 and/or a server 505. The portable detection system may send data associated with the optical measurement to any or all of the receiving device 501, the further receiving device 503 and the server 505. Any or all of the receiving device 501, the further receiving device 503 and the server 505 may determine the change in power at the resonant wavelength that corresponds with the pathogen. It will be understood that the receiving device 501 and/or the further receiving device 503 may be any suitable device such as a mobile telephone, a tablet device, a laptop, a computer or any suitable smart ware (e.g. a smartwatch, smart glasses etc.) etc.

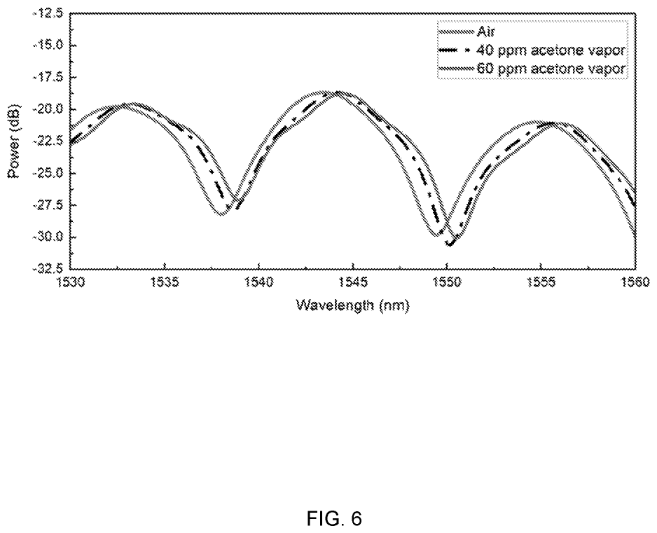

FIG. 6 shows an example optical power output signal generated by a portable detection system that has been set up to detect acetone vapour in an air sample. As can be seen, a change in power intensity (y-axis) is measured at different wavelengths (x-axis) and the power intensity at resonant wavelengths is determined, i.e. based on the peaks and troughs of the power intensity measurements. Therefore, a microcontroller can determine, based on the data sets provided, whether a certain pathogen is present or not in the sample.

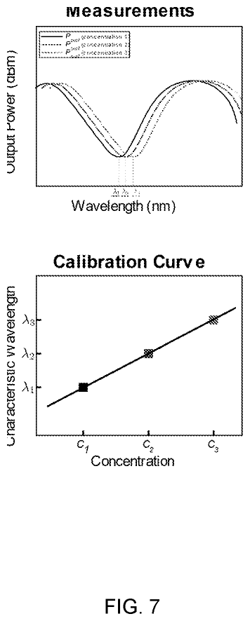

FIG. 7 shows an example of a calibration curve for output power intensity with regard to the concentration of the pathogen. The bottom plot illustrates the linear relationships between the characteristic wavelength of the resonant wave and the concentration of the pathogen, where the wavelength increases with the concentration. The top plot shows that the shift in output power intensity can be measured with different characteristic wavelengths. Therefore, the portable detection systems can be calibrated with the corresponding concentration.

### INDUSTRIAL APPLICABILITY

The arrangements described are applicable to the virus or pathogen sensing industries.

The foregoing describes only some embodiments of the present invention, and modifications and/or changes can be made thereto without departing from the scope and spirit of the invention, the embodiments being illustrative and not restrictive.

In the context of this specification, the word “comprising” means “including principally but not necessarily solely” or “having” or “including”, and not “consisting only of”. Variations of the word “comprising”, such as “comprise” and “comprises” have correspondingly varied meanings.

1. A portable detection system for detecting a pathogen or virus, the portable detection system comprising:
at least one light source for generating an input optical signal;
at least one optical sensor comprising at least one optical fibre having at least one tapered sensing region with at least one coating that reacts with a pathogen being detected, wherein the optical sensor is arranged to receive the input optical signal, communicate the input optical signal through the tapered sensing region and communicate an output optical signal from the tapered sensing region;
at least one photodetector for detecting power intensity of the output optical signal, wherein the photodetector is arranged to generate an output signal based on a change in power intensity of the output power at a resonant wavelength that corresponds with the pathogen; and
at least one microcontroller for controlling the light source, optical sensor and photodetector;
wherein the portable detection system is arranged to cause an output to be generated based on the output signal, wherein the output is associated with the pathogen being detected.

2. The portable detection system of claim 1 further comprising an output interface module, wherein the microcontroller is arranged to determine the change in power intensity at the resonant wavelength and generate the output based on the determined change in power intensity, and
the output interface module is arranged to generate one or more of a visual, audible and haptic output based on the determined change in power intensity.

3. The portable detection system of claim 1 or 2 further comprising a communication module for communicating a receiving device signal associated with the output to at least one receiving device.

4. The portable detection system of claim 3 wherein the receiving device is arranged to receive the receiving device signal, determine the change in power intensity at the resonant wavelength, and based on the determined change in power intensity, generate one or more of a visual, audible and haptic output based on the determined change in power intensity.

5. The portable detection system of claim 4, wherein the receiving device further comprises an output interface module,
the output interface module arranged to generate, based on the receiving device signal, one or more of a visual, audible and haptic output based on the output.

6. The portable detection system of any one of claims 1 to 5, wherein the coating comprises one or more of a phage, nanomaterials, copper complex, proteins and enzymes.

7. The portable detection system of any one of claims 1 to 6, wherein the optical sensor is a connectable and detachable optical probe.

8. The portable detection system of any one of claims 1 to 7, wherein the light source comprises a super luminescent light-emitting diode.

9. The portable detection system of claim 1, further comprising a power source for providing power to one or more of the light source, optical sensor, photodetector, and microcontroller.

10. A detachable optical probe for use in the portable detection system of claim 1, the optical probe comprising a sensor interface for interfacing with the portable detection system, and an optical sensor, wherein the optical sensor comprises an optical fibre having at least one tapered sensing region with at least one coating that reacts with a pathogen being detected, wherein the optical sensor is arranged to receive an input optical signal generated by at least one light source, communicate the input optical signal through the tapered sensing region and communicate an output optical signal from the tapered sensing region.

11. The detachable optical probe of claim 10 further comprising the at least one light source for generating the input optical signal.

12. The detachable optical probe of claim 10 further comprising at least one photodetector for detecting output power of the output optical signal, wherein the photodetector is arranged to detect a change in power intensity of the output power at a resonant wavelength that corresponds with the pathogen.
